SEQUENCE LISTING (27 sequences)

SEQ ID NO: 1 - DNA, 22 bp, Artificial sequence
  Synthetic oligonucleotide
cagctgaggataggacattcga

SEQ ID NO: 2 - DNA, 21 bp, Artificial sequence
  Synthetic oligonucleotide
tcagaactcacctgttagacg

SEQ ID NO: 3 - DNA, 36 bp, Artificial sequence
  Synthetic oligonucleotide
atcagcaccctggctgatcttagtgtcaggatacgg

SEQ ID NO: 4 - DNA, 41 bp, Artificial sequence
  Synthetic oligonucleotide
atcagcaccctggctgattagtacgcttctactccctcttg

SEQ ID NO: 5 - DNA, 24 bp, Artificial sequence
  Synthetic oligonucleotide
actagagctgagacatgacgagtc

SEQ ID NO: 6 - DNA, 22 bp, Artificial sequence
  Synthetic oligonucleotide
acgacgtgtgaccagtcaacat

SEQ ID NO: 7 - DNA, 24 bp, Artificial sequence
  Synthetic oligonucleotide
actagagctgagacatgacgagtc

SEQ ID NO: 8 - DNA, 22 bp, Artificial sequence
  Synthetic oligonucleotide
acgacgtgtgaccagtcaacat

SEQ ID NO: 9 - RNA, 24 nt, Artificial sequence
  Synthetic oligonucleotide
acuagagcugagacaugacgaguc

SEQ ID NO: 10 - RNA, 22 nt, Artificial sequence
  Synthetic oligonucleotide
acgacgugugaccagucaacau

SEQ ID NO: 11 - DNA, 21 bp, Artificial sequence
  Synthetic oligonucleotide
ccgtgctagaaggaaacacgc

SEQ ID NO: 12 - DNA, 15 bp, Artificial sequence
  Synthetic oligonucleotide
gtaccgcagccagtc

SEQ ID NO: 13 - DNA, 18 bp, Artificial sequence
  Synthetic oligonucleotide
tatatgatggtaccgcag

SEQ ID NO: 14 - DNA, 21 bp, Artificial sequence
  Synthetic oligonucleotide
ccgtgctagaaggaaacacgc

SEQ ID NO: 15 - DNA, 22 bp, Artificial sequence
  Synthetic oligonucleotide
tatatgatggtaccgcagccag

SEQ ID NO: 16 - DNA, 42 bp, Artificial sequence
  Synthetic oligonucleotide
acgatgactgacggtcatcgttcagaactcacctgttagacg

SEQ ID NO: 17 - DNA, 43 bp, Artificial sequence
  Synthetic oligonucleotide
acgatgactgacggtcatcgttatatgatggtaccgcagccag

SEQ ID NO: 18 - DNA, 24 bp, Artificial sequence
  Synthetic oligonucleotide
caactgtggttaaagcaatagtgt

SEQ ID NO: 19 - DNA, 24 bp, Artificial sequence
  Synthetic oligonucleotide
gcacagattctgagtaaccataat

SEQ ID NO: 20 - DNA, 107 bp, Artificial sequence
  Synthetic oligonucleotide
aagaactatattgtctttctcgcatgtcctatcctcagctgtgatcatcagaactcacctgttagacgccaccagctccatccactcagtgtgattccaccttctcc

SEQ ID NO: 21 - DNA, 42 bp, Artificial sequence; identical to SEQ ID NO: 16
  Synthetic oligonucleotide

SEQ ID NO: 22 - DNA, 21 bp, Artificial sequence
  Synthetic oligonucleotide
ccgtgctagaaggaancacgc

SEQ ID NO: 23 - DNA, 42 bp, Artificial sequence
  Synthetic oligonucleotide
acgatgactgacggtcatcgtccgtgctagaaggaancacgc

SEQ ID NO: 24 - DNA, 42 bp, Artificial sequence
  Synthetic oligonucleotide
acgatgactgacggtcatcgtccgtgctagaaggaaacacgc

SEQ ID NO: 25 - DNA, 43 bp, Artificial sequence; identical to SEQ ID NO: 17
  Synthetic oligonucleotide

SEQ ID NO: 26 - DNA, 21 bp, Artificial sequence
  Synthetic oligonucleotide
ccgtgctagaaggaancacgc

SEQ ID NO: 27 - DNA, 42 bp, Artificial sequence; identical to SEQ ID NO: 23
  Synthetic oligonucleotide

(19) United States
(12) Patent Application Publication
(10) Pub. No.: US 2004/0115674 A1
(43) Pub. Date: Jun. 17, 2004

(54) Suppression of non-specific nucleic acid amplication

(76) Inventors: Tim Knott, Buckinghamshire (GB); Clifford Smith, Buckinghamshire (GB); Judith Pickering, Buckinghamshire (GB); Terek Schwarz, Buckinghamshire (GB)

Correspondence Address: AMERSHAM BIOSCIENCES, PATENT DEPARTMENT, 800 CENTENNIAL AVENUE, PISCATAWAY, NJ, 08855, US

(21) Appl. No.: 10/466,580

(22) PCT Filed: Jan. 15, 2002
(86) PCT No.: PCT/GB02/00144

(30) Foreign Application Priority Data
Jan. 19, 2001 (GB) 0101397.8

(51) Int. Cl. C12Q 1/68; C12P 19/34

(52) U.S. Cl. 435/6; 435/91.2

(57) ABSTRACT

The invention discloses methods of reducing background signal in nucleic acid amplification reactions by the use of primers in the case of isothermal amplification which include at least one modification selected from a nucleotide analogue, a hairpin loop at the 5′ end of the primer, a ribonucleotide or a fluor or quencher. For more general nucleic acid amplification reactions the primer includes at least two of the modifications.

### FIELD OF THE INVENTION

The disclosed invention applies to the field of assays for detection of analytes, and specifically the field of nucleic acid amplification and detection.

### BACKGROUND OF THE INVENTION

A number of methods are known that enable sensitive diagnostic assays based on nucleic acid detection. Many involve exponential amplification of the nucleic acid target or probe sequences. They include the polymerase chain reaction (PCR), ligase chain reaction (LCR), self-sustained sequence replication (3SR), nucleic acid sequence based amplification (NASBA), strand displacement amplification (SDA), and rolling circle amplification (RCA) Lizardi, et al (1998). Nature Genetics 19:225-232, Lizardi, P. M., & Ward, D. C. (1997) Nature Genetics 16:217-218, Fire, A., & Xu, S-Q. (1995) Proc. Natl. Acad. Sci. USA 92: 4641-4645, Liu, D., et al (1996) J. Am. Chem. Soc. 118: 1587-1594 and Zhang et al (1998) Gene 211: 277-285 and WO 97/19193). All display good sensitivity, with a practical limit of detection of about 10-100 target molecules.

Accuracy and robustness determine the usefulness of any nucleic acid based assay, particularly when only a few molecules of target are present. It is vital that the process is highly specific. Amplification of untargeted sequences or nontarget directed amplification impacts severely upon assay reliability. Each of the above methods is capable of generating and amplifying non-specific or spurious background signals.

A frequent source of background amplification in PCR reactions is the hybridisation of a primer to regions of input DNA that share some homology with the targeted sequence. If the 3′ end of a primer has sufficient homology to the untargeted region then it can be amplified in a DNA polymerase reaction. In some instances the resultant, spurious primer extension product may be further amplified. An additional cause of background is attributable to intra- or inter-strand primer annealing, leading to so-called ‘primer-dimer’ artifacts. In extreme cases side reactions can predominate and may totally inhibit or mask amplification of the targeted sequence.

RCA is applicable to the amplification and detection of specific analytes, such as nucleic acids, proteins and other biomolecules in a sample. Being an isothermal method, RCA, eliminates the need for thermal cycling used in alternative processes such as PCR and, unlike PCR, the target molecule is not amplified. Thus, propagation of polymerase-induced mutations is minimised.

As already mentioned above, several different formats of rolling circle amplification have been described. The common element is amplification from a small, single stranded, circular DNA probe that is formed via chemical or enzymatic ligation of a linear pre-circle hybridized to a target molecule, Baner J., et al (1998) Nucl. Acids Res. 26: 5073. Ligation of the linear nucleic acid probe generates circular probe molecules proportional in number to the amount of target sequence present in a sample. Rolling circle replication of the circularired probe is an isothermal process mediated via a single primer and a processive, strand-displacing DNA polymerase, resulting in up to 10⁴-fold amplification per hour. The reaction kinetics are linear and hence this process has been termed linear RCA [LRCA].

In an extension of LRCA additional oligonucleotide primers are employed to replicate the primary, single stranded amplification product. This technique is known variously as hyper-branched, cascade or exponential RCA [ERCA] (Lizardi (supra) and Thomas, et al (1999) Arch. Pathol. Lab. Med. 123: 1170. Here amplification proceeds with geometric kinetics, directing synthesis of branched, double stranded DNA product at rates in excess of 10⁹-fold The first primer hybridises to its complementary region on the probe backbone. In the presence of a strand-displacing DNA polymerase, the primer is extended, eventually displacing itself at its 5′ end once one complete revolution of the circularised probe is made. Continuing polymerisation and strand displacement result in the generation of a long, single stranded, concatameric DNA copy of the original probe circle. This single stranded RCA product, contains binding sites for the second primer. The second primer binds to each tandem repeat of the first strand product. As these multiple priming events elongate, they too initiate strand displacement, in turn creating single-stranded DNA products which expose further binding sites for the first amplification primer. An extensive, hyper-branched structure is built up which contains many replication forks. Self-propagating, strand-displacement results in the release of double stranded DNA fragments from this replication complex. These displaced DNA molecules accumulate as a nested population of fragments displaying sizes that are multiples of the circle unit length.

RCA probes or pre-circles consist of a linear, 5′-phosphorylated oligonucleotide, usually between 60-120 bases in length. Sequences at the 5′ and 3′ ends of the probe are complementary to the target region such that, when hybridized to its target, the probe ends are juxtaposed. A dual hybridization event combined with the stringent base pairing requirements of a thermostable DNA ligase confers a high degree of target specificity. Located between the target-specific probe arms is a unique sequence that provides binding sites for RCA amplification primers. Probes can be made to distinguish between two alleles that may be present in the target nucleic acid sequence. The terminal 3′ base is varied to complement each of the two possible alleles at the polymorphic site. Probe design and ligation conditions can be optimized to allow allelic discrimination directly in the complex sequence context of genomic DNA without the need for pre-amplification of the target region.

It is possible to specifically amplify individual circularized probes in a mixture by virtue of their unique backbone sequence. Each probe can be amplified using its specific primer [LRCA] or pair of primers [ERCA]. Amplified probe sequences can be detected and quantified by conventional methods such as fluorescent labels, enzyme-linked detection systems, antibody-mediated label detection, and detection of radioactive labels. One approach, based upon fluorescent detection, utilises Amplifluor™ technology Nazarenko, et al (1997) Nucl. Acids Res. 25: 2516-2521. Amplifluor™ detection primers carry a hairpin stem-loop on their 5′ end, labeled near the base of the stem with a fluorophore and a quencher. In one condition the fluor and quencher are in sufficient proximity for efficient fluorescence quenching to occur. When an Amplifluor™ primer is used as the second ERCA amplification primer, it becomes incorporated into the double-stranded DNA products. As the DNA polymerase copies the Amplifluor™ primer the it unfolds and synthesizes the complement of the stem-loop structure, thus linearizing the sequence and physically separating the fluorophore and quencher and resulting in a fluorescent end product. Use of several Amplifluor™ primers each labeled with a different fluorophore facilitates multiplexed detection in a single RCA reaction.

One problem affecting RCA reactions is circle-independent or target-independent DNA synthesis. It is reported that one form of circle-independent artifact in dual-primer RCA reactions is reduced by strategies designed to eliminate excess un-ligated probe. For example, in WO 00/36141 Hafner et al suggest that RCA backgrounds can arise from alternative amplification reactions that utilize linear probe molecules.

We have observed that under certain conditions artifactual RCA products can accumulate to high levels in the absence of circularized probes and or target DNA. The problem is most likely to arise in ERCA reactions containing two primers although, as illustraed herein, non-specific amplification can initiate from a single primer. The likelihood of artifacts increases significantly in multiplex assays utilizing 4 or more different RCA primers. When cloned and sequenced, the circle-independent amplification products are found to be predominantly multimers of head-to-tail primer repeats. In addition to primer sequences, each repeat unit may also contain one or more sequence segments of up to 15 bases not derived from either the target or probe but thought to originate from bacterial DNA contamination commonly associated with commercial sources of molecular biology enzymes.

Many non-separation based detection strategies will falsely score these products as positive results. It is therefore particularly important for homogeneous assay systems that DNA synthesis does not occur in the absence of legitimate circularized probe molecules.

The prior art documents several attempts at reducing non-specific events in amplification and hybridization reactions by including various modifications to the primer(s). See for example EP 866071, WO 01/25478, WO 98/13527. These methods exert their effect through increasing the specificity of primer-target interaction, either through lowering Tm by disrupting hydrogen bonding (EP 866071 and WO 98/ 13527) or by shortening the primer but maintaining its Tm using high affinity analogues (WO 01/25478). In EP 866071 modifications are placed within 4-6 bases of the primer 3′ end, within the polymerase footprint, for maximum effectiveness. In contrast, the current invention expressly avoids modifying this region so as not to adversely affect priming efficiency.

Stump et al (Nucleic Acids Res. 27, 4642-4648 (1999)) have used primers modified with RNA analogues or abasic sites to eliminate artifacts in thermocycled DNA sequencing reactions. The authors demonstrated that such primers could not be used for exponential amplification reactions because after initial extension the DNA polymerase cannot copy the modified primer during subsequent reaction cycles. Whereas Stump et al were unable to show exponential amplification with primers of this design, we show here that primer copying is not an absolute requirement for exponential RCA and that non-replicatable primers can be used effectively to block artifactual amplification in isothermal RCA reactions.

### SUMMARY OF THE INVENTION

This invention improves the sensitivity of nucleic acid based amplification strategies, reducing or eliminating non-specific background signals arising from primer multimers. This is achieved by blocking or impairing the ability of primers to serve as effective templates for DNA synthesis.

The invention provides a nucleic acid probe or primer, a region of which is modified so as to inhibit or block the molecular interactions that generate primer-based artifacts. In one feature of the invention the modification takes the form of a palindrome that forms a stable hairpin loop structure at the assay temperature. An additional feature is the covalent attachment of chemical moieties such as, but not limited to, dyes. A further modification involves the inclusion of nucleoside analogues within primers.

The invention also provides methods and reagents that suppress non-specific background amplification.

### DETAILED DESCRIPTION OF THE INVENTION

The invention provides a method of suppressing background signal in an isothermal nucleic acid amplification reaction wherein at least one of the primers used comprises at least one of

a nucleotide analogue

a haipin loop at the 5′ end of the primer

a ribonucleotide

a fluor or quencher.

Another aspect of the invention for suppressing background signal in a nucleic acid amplification reaction requires the presence in at least one of the primers of at least two of

a nucleotide analogue

a hairpin loop at the 5′ end of the primer

a ribonucleotide

a fluor or quencher.

Non-specific amplification is a problem in nucleic acid amplification schemes that utilize one or more oligonucleotide primers. A mechanism of non-specific amplification has been identified in RCA reactions that is independent of legitimate circular probe molecules and will also arise in the absence of linear probe and target molecules. This type of artifactual DNA synthesis generates a nested set of predominantly double stranded DNA molecules ranging between 50 base pairs to more than 20 kilobase pairs in size and forming a characteristic ladder of products that is frequently indistinguishable from that of a genuine, circle-mediated RCA reaction. DNA sequence analysis of the non-specifically amplified material suggests that it may originate due to a continuous series of self-propagating strand displacement and primer extension events. A single primer, four deoxynucleoside tiphosphates and a DNA polymerase are sufficient to support the synthesis of several micrograms of high molecular weight DNA in a 1-2 hour isothermal reaction. No probe molecules or added target molecules are necessary for non-specific amplification to occur.

A robust and reliable nucleic acid amplification assay hinges upon the principle that no product is formed in the absence of a specific target molecule. Hence it is vital to prevent non-specific amplification of the type described.

One aspect of the invention is to provide a method for suppressing the synthesis of non-specific products in a nucleic acid amplification reaction. This is accomplished without reducing the generation of specific targeted products. In this way the signal to noise ratio, sensitivity and reliability of the method are increased.

In particular, the invention provides a method of RCA in which background DNA synthesis due to non-specific amplification is inhibited when circular probe molecules are not present The invention is useful in all modes of RCA including single primer, dual primer and multiple primer amplification reactions.

In one embodiment non-specific amplification is inhibited by the use of one or more oligonucleotide primers that contain at least one nucleotide analogue. In LRCA and ERCA it is not essential for the complement of a primer to be made in order for the amplification reaction to be sustained. Thus analogues which render the primers poor templates for polymerase enzymes can be employed to suppress primer self-amplification. Suitable analogues may be positioned at any point in the primer sequence but preferably the 6 positions closest to the 3′ terminus should be unmodified so as not to impact priming efficiency. Examples of nucleotide analogues and related modifications that have been found to be effective include, but are not limited to, locked nucleic acid bases [LNA] (Singh et al (1998) Chem. Commun. 455-456), 2′-O-Methyl RNA bases, substituted 5-nitroindole (WO97/28176), abasic sites and RNA. Single or multiple sites may be modified In certain instances it is desirable to modify adjacent or consecutive bases in order to minimise polymerase read-though. As polymerases differ in their ability to copy templates bearing nucleoside analogues it is necessary to determine empirically the optimal type position and frequency of modified bases. Examples of polymerases that may be used include, but are not limited to, phi 29 DNA polymerase, ThermoSequenase™ II, delta, Thermoanaerobacter thermohydrosulfuricus DNA polymerase, Bst DNA polymerase, Phi 29 DNA polymemse and Sequenase™ T7 DNA polymerase. Preferably Bst DNA polymerase or Phi 29 DNA polymerase are used.

In a further embodiment, non-specific amplification may be inhibited by the use of one or more oligonucleotide primers with a 5′ region capable of intra-strand base pairing in such a way as to form a duplex stem and loop structure. Suitable primers are composed of four contiguous sequence elements S1, S2, S3 and S4. S1 being at the 5′ terminus and S4 at the 3 terminus of the primer. S1 is the reverse complement of S3. S2 is a spacer region. S4 may be either complementary to or identical to a region of the circular probe molecule. Preferably S1 and S3 are between 4-12 bases long. S2 should be greater than 3 bases long, preferably 5-20 bases long. S4 is of a length calculated to provide a Tₘ equal to the temperature of the amplification reaction. The sequences of S1, S2 and S3 are chosen such that the ΔG of the desired secondary structure is suitably at least 6 kCal and more preferably at least 10 kCal greater than that of any alternative structure. Established guidelines for designing primers for use in nucleic acid amplification reactions are followed in addition to the specific requirements detailed here. Non-specific amplification by ThermoSequenase™ II in dual primer RCA reactions was inhibited when one of a pair of primers carried a 5′ hairpin as described. The same primers lacking a hairpin synthesized large amounts of high molecular weight artifacts under identical conditions. Additionally, ThermoSequenase II was unable to amplify background by RCA in the presence of a single primer when that primer carried a 5′ hairpin. Non-specific RCA by Bst DNA polymerase using a single hairpin primer was not suppressed but it was found that if a fluorophore and fluorescence-quenching moiety were coupled to the same hairpin primer then non-specific amplification was suppressed. Accordingly, this invention also provides a method for inhibiting non-specific amplification by use of primers having the structure of Amplifluor™ primers Nazarenko et al (supra). Fluorophores that have been found useful in this regard include, but are not limited to, 6-FAM Fluorescein, Cy3 and TET. Quenchers that may be used include DABCYL, DABSYL and Methyl Red.

In yet a further embodiment, non-specific amplification can be suppressed during dual and multiple primer ERCA by use of a linear or a hairpin primer bearing nucleotide analogues in combination with an Amplifluor™ primer bearing nucleotide analogues.

Although the examples cited here reference RCA, it will be appreciated that alternative amplification schemes such as SDA and LAMP (Notomi, et al (2000) Nucl. Acids Res. 28: (12) e63) that utilize similar DNA polymerases and primers in isothermal conditions may exhibit similar modes of non-specific amplification to the type described This invention is thus equally applicable to these techniques.

It is anticipated that the invention is also applicable to any nucleic acid amplification method in which copying of primer molecules by a polymerase enzyme may contribute to non-specific amplification and thereby leading to spurious reaction products.

### EXAMPLES

### Example 1

### Non-Specific Amplification.

DNA polymerases can synthesise double-stranded, high molecular weight DNA under isothermal conditions if given just primers and the four common deoxynucleoside triphosphates (dATP, dCTP, dGTP and dTTP).

20 μl reactions containing 20 mM Tris-HCl pH 8.8, 0.1% v/v Triton X-100, 10 mM KCl, 2 mM MgSO₄, 400 μM dNTP, 8 units Bst DNA polymerase [New England Biolabs] and 1 μM primer were incubated at 60° C. for 90 minutes. Ficoll/Orange-G loading dye was added to each reaction and 10 μl was run on a 2% high resolution agarose gel [Sigma A-4718] in 90 mM Tris-Borate/EDTA buffer pH8.3 for ˜1.5 hours at 150 volts. The gel was stained in a 1:20,000 aqueous dilution of Vistra Green [Amersham Pharmacia Biotech] for 15 minutes then scanned on a Molecular Dynamics FluorImager-595 using 488 nm excitation and 530 nm emission filters.

A spectrum of DNA products was generated from primers and dNTPs alone by Bst DNA polymerase. The results showed a ladder pattern with approximately 40 bp periodicity when the reaction contained both primers #1 and #2. When reactions contained only primer #1 or primer #2, a complex series of fragments was made, which range from primer length to material so large that it does not enter the gel. Reactions with Bst DNA polymerase and dNTPs only (no primers) did not produce high molecule weight product. Reactions containing primers #1 or #2 and dNTPs but no polymerase again did not produce high molecule weight product.

### Example 2

### Suppression of Non-Specific Amplification by Primers Containing LNA.

As the number of different primers in an RCA reaction rises so the risk of non-specific amplification increases. Duplex ERCA reactions were carried out in which each reaction contained two distinct pre-formed circular DNA probe molecules. Two unique, specific RCA primers were included for each circular DNA. One of each pair of primers was an Amplifluor™ primer and the other was either a linear DNA primer or a DNA/LNA chimeric primer.

Serial dilutions containing both gel-purified, circularized probes were amplified by ERCA for 2 hours at 65° C. in a 20 μl reaction containing 20 mM Tris-HCl pH 8.8, 0.1% v/v Triton X-100, 10 mM KCl, 10 mM (NH4)₂SO₄, 2 mM MgSO₄, 400 μM dNTP, 8 units Bst DNA polymerase, 0.4 μM FAM-dabcyl Amplifluor primer #3, 0.3 μM Cy3-dabcyl Amplifluor primer #4 and either 0.4 μM DNA primers #5 and #6 or 0.4 μM LNA/DNA chimeric primers #7 and #8.

After ERCA, 2 ul of tracking dye [50% w/v Ficoll F400, 1% w/v Orange-G, 50 mM EDTA] was added and the samples were electrophoresed on a 3% high-resolution agarose gel in 90 mM Tris-borate/EDTA buffer for 2½ hours at 125 volts. The gel was scanned twice in a Molecular Dynamics FluorImager 595 using an excitation wavelength of 488 nm and recording emission a both 530 nm and 570 nm. The two individual colour images were overlaid.

The gel was then stained by immersion in a 1:20,000 aqueous dilution of Vistra Green and re-scanned with 488 nm excitation and 530 nm emission filters to visualise both fluorescently labelled and unlabelled DNA products.

Reactions containing 10⁶, 10⁵, 10⁴, 10³, 10², 10¹, 10⁰, or 0 circles were performed with either DNA primers or with LNA/DNA chimeric primers of identical base sequence.

When 10⁵ or more circular probe molecules were present all reactions gave the expected fluorescent-labelled product ladders. At 10⁴ copies of circular probe and below, both non-specific fluorescent and non-fluorescent amplification products appeared in those reactions that had DNA primers only. Reactions amplified in the presence of DNA/LNA primers showed no non-specific fluorescent or non-fluorescent ladders—only correct fluorescent products were formed.

### Example 3

### Suppression of Non-Specific Amplification by Primers Containing RNA.

Most DNA polymerases have minimal detectable levels of reverse transcriptase activity. Modes of non-specific amplification dependent upon primer copying in RCA reactions can be significantly reduced or eliminated where RCA primers are comprised wholly, or partly, of RNA.

30 μl ligation reactions were prepared containing 20 mM Tris-HCl pH8.3, 25 mM KCl, 10 mM MgCl₂, 0.01% v/v Triton X-100, 1.5 mM NAD⁺, 100 nM open circle probe, 1 unit Tth DNA ligase and 10³, 10⁵ or 10⁷ molecules of a PCR-amplified DNA target molecule. Reactions were denatured at 95° C. for 3 minutes then incubated at 65° C. for 60 minutes and cooled to 4° C.

1/10^{th} of each ligation reaction was subjected to ERCA in 20 μl containing 200 mM Tris-acetate pH 8.5, 1% v/v Triton X-100, 100 mM (NH₄)₂SO₄, 400 μM dNTP, 8 units Bst DNA polymerase, 0.4 μM FAM-dabcyl Amplifluor primer #3, 0.3 μM Cy3-abcyl Amplifluor primer #4 and either 0.4 μM DNA primers #5 and #6 or 0.4 μM RNA primers #9 and #10. Reactions were incubated for 90 minutes at 65° C. Gel analysis and imaging were as described in Example 2.

ERCA reactions containing DNA primers showed the anticipated fluorescent amplification products when 10⁷ PCR target molecules were used for probe ligation and circularization. No fluorescent signal was seen for 10⁵ or 10³ target molecules. In addition, all reactions with DNA primers generated substantial amounts of non-specific, non-fluorescent material.

Reactions that contained RNA versions of DNA primers showed no non-specific or non-fluorescent products. RNA primers gave only specific fluoreccent product ladders in both 10⁷ and 10⁵ target copies. Control ERCA reactions having no targets no DNA ligase or no ligase reaction added were negative as expected.

RNA primers suppressed non-specific background amplification but did not inhibit rolling circle amplification of circular probe molecules.

### Example 4

### Suppression of Non-Specific Amplification by a Primer With a 5′ End Hairpin Loop.

It was found that non-specific amplification involving primer copying in a dual primer reaction could be suppressed if one of the primers has a 5′ end hairpin loop and the reaction is carried out with ThermoSequenase™ II DNA polymerase.

30 μl reactions were prepared containing 30 μM each RCA primer, 250 mM Tris-acetate pH8.0, 17.5 mM magnesium acetate, 125 mM potassium glutamate, 5% v/v glycerol, 8 nM dNTPs and 20 units of ThermoSequenase™ II. Reactions were heat denatured at 95° C. for 3 minutes and then incubated for 60 minutes at 68° C. Amplification products were analysed on gels as described in Example 1. Reactions contained two primers #11 and #12, #11 and #13, #1 and #2, #14 and #15, #1 and #16, or #14 and #17.

Reactions with pairs of linear primers resulted in a 50 base pair ladder of non-specific amplification products. Substitution of one member of a pair of linear primers by one of identical priming sequence plus a 5′ hairpin structure (8 base pair stem and 5 base unpaired loop) prevented non-specific amplification.

### Example 5

### Suppression of Non-Specific Amplification by Primers Containing Substituted 5-Nitroindole Base Analogues

5-amino-pentanoic acid{4-[1-(4-hydroxy-5-hydroxymethyltetrahydrofuran-2-yl)-5-nitro-1H-indol-3-yl]-butyl}-amide, a 5-nitroindole base analogue with a C₆ spacer arm at the 3 position was synthesized as a phosphoramidite by methods described in WO97/28176. It was shown that DNA polymerases are unable to read past this base analogue when it is present in a single stranded DNA template. DNA primers containing substituted 5-nitroindole were prepared and their ability to suppress non-specific amplification was demonstrated.

Experiments were carried out according to the method outlined in Example 1. Reactions containing a single unmodified primer produced a characteristic ladder of artifactual products. In the presence of a primer (either linear or with a 5′ hairpin) that was modified internally with a single substituted 5-nitroindole six bases from the 3′ terminus, no non-specific amplification was observed.

### Example 6

### Suppression of Non-Specific Amplification by an Amplifluor Primer

A region corresponding to the putative nucleotide (ATP)-binding folds of the Human cystic fibrosis gene was PCR-amplified using primers 11i-5 and 11i-3 (sequences #18 and #19) as described by Kerem, B-S. et al, Proc. Natl. Acad. Sci. USA. 87: 8447.

A series of 20 μl ligation reactions were set up containing from 10⁹-10⁵ copies of homozygous normal or homozygous G542X mutant PCR fragment, 10 nM G542X open circle probe (sequence #20), 20 mM Tris-HCl pH8.3, 25 mM KCl, 10 mM MgCl₂, 0.01% v/v Triton X-100, 1.5 mM NAD⁺ and 1 unit Tth DNA ligase. After heat denaturation at 95° C. for 3 minutes ligation mixes were incubated for 60 minutes at 65° C.

30 μl ERCA reactions contained 2 μl ligation mixture, 20 units ThermoSequenase™ II, 250 mM Tris-acetate pH8.0, 17.5 mM magnesium acetate, 125 mM potasium glutamate, 5% v/v glycerol, 8 mM dNTPs, 30 μM primer #1 and either 30 μM primer #2 or 30 μM Amplifluor™ primer #21. Samples were heated to 95° C. for 3 minutes and then incubated at 68° C. for 60 minutes. Gel analysis and imaging were as described in Example 2.

Open circle probe was ligated in the presence of either matched or mismatched PCR target DNA. Matched means that the open circle probe is the exact complement of the target and that ligation should occur. Mismatched indicates that little or no ligation and amplification should take place. Target DNA was present at 10⁹, 10⁷ and 10⁵ copies per ligation. Circularized probes were amplified by ERCA using Thermosequenase™ II at 68° C. for 60 minutes. Reactions contained both one linear and one Amplifluor™ primer or two linear RCA primers. For linear primers, there was substantial non-specific amplification with matched probe/target combinations below 10⁹ target copies and in all mismatched reactions. Substitution of the linear primer for an Amplifluor™ primer completely inhibited background amplification, leaving only a ladder of specific products. The Amplifluor™ reaction products are larger due to the increased primer length and appear blurred on native agarose gels due to unresolved secondary structures.

### 7

### Suppression of Non-Specific Amplification by a Combination of Amplifluor Primers and Analogue-Modified 5′ Hairpin Primers.

Whereas a 5′ hairpin structure was sufficient to block non-specific amplification of a single primer by ThermoSequense II it was not effective for Bst DNA polymerase. However, Bst DNA polymerase was unable to amplify a single primer when, in addition to a 5′ hairpin, the priming region contained one or base modifications that prevented read through by the enzyme. Modifications found to be effective included abasic sites and substituted 5-nitroindole. Individual Amplifluor primers, without base modifications, were also refractory to amplification by Bst polymerase.

When two or more primers were combined non-specific amplification could be prevented only if (1) both carried a hairpin and modified bases or (2) if the first carried a hairpin and modified bases and the second was an Amplifluor primer or (3) if both primers were Amplifluor primers.

Primer #14 was an unmodified linear primer, primer #22 had the same sequence but with an abasic site 6 bases from the 3′ terminus and primer #23 was similar to #22 but with the addition of a 5′ end hairpin. Primer #25 was an Amplifluor primer. Amplification reactions and gel analyses were as described in Example 1.

Separate reactions utilizing

primers #14 and #25.

primers #22 & #25.

primers #23 & #25.

primers #24 & #25.

primers #16 & #25.

no primers, Bst DNA polymerase and dNTPs only.

primers #16 & #17, linear primers only.

primers and dNTPs only, no Bst DNA polymerase.

were performed. Apart from the two negative controls only one reaction failed to generate any non-specific amplification products. This reaction contained one hairpin primer modified at position -6 with an abasic site and one FAM-dabcyl Amplifluor primer,

Non-specific primer pair amplification in dual primer ERCA reactions involving a strand displacing DNA polymerase can be reduced if each of the primers has either a 5′-end hairpin plus base analogues in the priming region or is an Amplifluor primer.

!Sequences?

| | | |
|---|---|---|
| #1 | 5′ CAGCTGAGGATAGGACATTCGA | |
| #2 | 5′ TCAGAACTCACCTGTTAGACG | |
| #3 | 5′ FAM-ATCAGCACCCTGGCTGAtCTTAGTGTCAGGATACGG | |
| | t = dabsyl-dT | |
| #4 | 5′ Cy3-ATCAGCACCCTGGCTGAtTAGTACGCTTCTACTCCCTCTTG | |
| | t = dabsyl-dT | |
| #5 | 5′ ACTAGAGCTGAGACATGACGAGTC | |
| #6 | 5′ ACGACGTGTGACCAGTCAACAT | |
| #7 | 5′ ACTAGAGCtgaGACATGACGAGTC | lower case letters are LNA bases |
| #8 | 5′ ACGACGTGtgaCCAGTCAACAT | lower case letters are LNA bases |
| #9 | 5′ acuagagcugagacaugacgaguc | lower case letters are RNA bases |
| #10 | 5′ acgacgugugaccagucaacau | lower case letters are RNA bases |
| #11 | 5′ CCGTGCTAGAAGGAAACACGC | |
| #12 | 5′ GTACCGCAGCCAGTC | |
| #13 | 5′ TATATGATGGTACCGCAG | |
| #14 | 5′-CCGTGCTAGAAGGAAACACGC | |
| #15 | 5′-TATATGATGGTACCGCAGCCAG | |
| #16 | 5′ ACGATGACTGACGGTCATCGTTCAGAACTCACCTGTTAGACG | |
| #17 | 5′-ACGATGACTGACGGTCATCGTTATATGATGGTACCGCAGCCAG | |
| #18 | 5′ CAACTGTGGTTAAAGCAATAGTGT | |
| #19 | 5′ GCACAGATTCTGAGTAACCATAAT | |
| #20 | 5′pAAGAACTATATTGTCTTTCTCGCATGTCCTATCCTCAGCTGTGATC | |
| | ATCAGAACTCACCTGTTAGACGCCACCAGCTCCATCCACTCAGTGTGAT | |
| | TCCACCTTCTCCTCCACCTTCTCC | |
| #21 | 5′-Cy3-ACGATGACTGACGGTCATCGtTCAGAACTCACCTGTTAGACG | |
| | t = dabsyl-dT | |
| #22 | 5′-CCGTGCTAGAAGGAAxCACGC | |
| | x = abasic | |
| #23 | 5′-ACGATGACTGACGGTCATCGTCCGTGCTAGAAGGAAxCACGC | |
| | x = abasic | |
| #24 | 5′-ACGATGACTGACGGTCATCGTCCGTGCTAGAAGGAAACACGC | |
| #25 | 5′ FAM-ACGATGACTGACGGTCATCGtTATATGATGGTACCGCAGCCAG | |
| | t = dabcyl-dT | |
| #26 | 5′-CCGTGCTAGAAGGAAxCACGC | |
| | x = 5-nitroindole + linker arm | |
| #27 | 5′-ACGATGACTGACGGTCATCGTCCGTGCTAGAAGGAAxCACGC | |
| | x = 5-nitroindole + linker | |

1) A method of suppressing background signal in an isothermal nucleic acid amplification reaction wherein at least one of the primers used comprises at least one of
a nucleotide analogue
a hairpin loop at the 5′end of the primer
a ribonucleotide
a fluor or quencher

2) A method of suppressing background signal in a nucleic acid amplification reaction wherein the primer comprises at least two of
a nucleotide analogue
a hairpin loop at the 5′end of the primer
a ribonucleotide
a fluor or quencher

3) A method according to claim 1 or 2 wherein the nucleotide analogue is selected from LNA, 2′-O-Methyl RNA, RNA, substituted 5′-nitroindole or abasic sites.

4) A method according to claim 1 to 3 wherein the nucleotide analogue is situated at least 6 bases from the primer 3′ terminus.

5) A method according to claim 1 to 4 wherein the amplification reaction is a rolling circle amplification method

6) A method according to claim 5 wherein the DNA polymerase used is selected from Bst DNA polymerase, ThermoSequenase™ II DNA polymerase, Phi 29 DNA polymerase or Sequenase™ T7 DNA polymerase.
